(19) United States
(12) Patent Application Publication
(10) Pub. No.: US 2021/0230696 A1
(43) Pub. Date: Jul. 29, 2021

(54) METHOD FOR PREDICTING THE RISK OF LATE-ONSET ALZHEIMER'S DISEASES

(71) Applicant: biotx.ai GmbH, Potsdam (DE)

(72) Inventors: Jörn Klinger, Potsdam (DE); Marco Schmidt, Berlin (DE)

(21) Appl. No.: 17/053,572

(22) PCT Filed: May 6, 2019
(86) PCT No.: PCT/EP2019/061550

§ 371 Date: Nov. 6, 2020

(30) Foreign Application Priority Data
May 7, 2018 (EP) 18171083.1

(51) Int. Cl. C12Q 1/6883

(52) U.S. Cl. CPC C12Q 1/6883; C12Q 2600/112; C12Q 2600/156; C12Q 2600/118

(57) ABSTRACT

A method for prognosis, risk assessment, risk stratification and/or diagnosis of a subject of developing late-onset Alzheimer's disease (LOAD), includes providing at least one sample isolated from the subject, and determining in the presence of ApoE4 and either rs1799931(G) or rs8192506(A), rs7653308(C), rs968529(C) and rs9658265(A). The presence or absence of these markers is indicative of a prognosis, a risk and/or a diagnosis of developing LOAD.

The invention relates to a method for prognosis, risk assessment, risk stratification and/or diagnosis of a subject of developing late-onset Alzheimer's disease (LOAD), comprising (i) providing at least one sample isolated from said subject, (ii) determining in said at least one sample the presence of (a) ApoE4 and at least one of (b) rs1799931(G) and/or (c) rs8192506(A), rs7653308(C), rs968529(C) and rs9658265(A), wherein the presence or absence of (a) ApoE4 and at least one of (b) rs1799931(G) and/or (c) rs8192506(A), rs7653308(C), rs968529(C) and rs9658265(A) is indicative of a prognosis, a risk and/or a diagnosis of developing LOAD.

### BACKGROUND OF THE INVENTION

Alzheimer disease (AD) is a progressive neurodegenerative disease associated with cognitive decline and is the most common form of dementia. Approximately 13% of people over the age of 65 and 45% over the age of 85 are estimated to have AD. In AD pathogenesis, an imbalance between the production and clearance of amyloid-β (Aβ) peptides in the brain results in accumulation and aggregation of Aβ. The toxic Aβ aggregates in the form of soluble Aβ oligomers, intraneuronal Aβ, and amyloid plaques injure synapses and ultimately cause neurodegeneration and dementia. The toxicity of Aβ seems to depend on the presence of microtubule-associated protein tau, the hyperphosphorylated forms of which aggregate and deposit in AD brains as neurofibrillary tangles.

There are 2 types of Alzheimer disease: early-onset and late-onset (LOAD). Early-onset familial AD, which typically develops before the age of 65 years and accounts for only a small portion (<1%) of AD cases, is primarily caused by overproduction of Aβ owing to mutations in either the APP gene or genes encoding presenilin 1 (PSEN1) or presenilin 2 (PSEN2), which represent essential components of the γ-secretase complexes responsible for cleavage and release of Aβ.

The majority of AD cases occur late in life (>60 years) and are commonly referred to as late-onset AD (LOAD). It is thought that multiple genetic and environmental risk factors are involved in LOAD pathogenesis, and overall impairment in Aβ clearance is probably a major contributor to disease development.

Genetically, the ε4 allele of the apolipoprotein E (ApoE) gene is the strongest risk factor for LOAD (Corder E H, et al. Gene dose of apolipoprotein E type 4 allele and the risk of Alzheimer's disease in late onset families. Science. 1993; 261:921-3; Huang Y, Mucke L. Alzheimer mechanisms and therapeutic strategies. Cell. 2012; 148:1204-22). The human ApoE gene exists as three polymorphic alleles—ε2, ε3 and ε4—which engender six different genotypes (ε2/ε, ε2/ε3, ε2/ε4, ε3/ε3, ε3/ε4 and ε4/ε4) and have a worldwide frequency of about 8%, 78% and 14%, respectively. However, the frequency of the ε4 allele is dramatically increased to ˜40% in patients with AD.

ApoE is composed of 299 amino acids and has a molecular mass of ˜34 kDa. Differences between the three ApoE isoforms are limited to amino acids 112 and 158, where either cysteine or arginine is present: ApoE2 (Cys112, Cys158), ApoE3 (Cys112, Arg158), and ApoE4 (Arg112, Arg158).

Genome-wide association studies have confirmed that the ε4 allele of APOE is the strongest genetic risk factor for AD. The presence of this allele is associated with increased risk for LOAD. A meta-analysis of clinical and autopsy-based studies demonstrated that, compared with individuals with an ε3/ε3 genotype, risk of AD was increased in individuals with one copy of the ε4 allele (ε2/ε4, OR 2.6; ε3/ε4, OR 3.2) or two copies (ε4/ε4, OR 14.9) among Caucasian subjects (Farrer, LaCLHJL., et al. Effects of age, sex, and ethnicity on the association between apolipoprotein E genotype and Alzheimer disease: A meta-analysis. JAMA. 1997; 278:1349-1356).

However, LOAD is a complex disease and the presence of ApoE4 only represents one of many potential risk factors. Different combinations of the homozygous or heterozygous presence of ApoE4 with other risk factors or protective factors, which may be both genetic and environmental, make it difficult to provide a good risk assessment for developing LOAD for an individual. ApoE-positive subjects represent a heterogeneous group of subjects comprising individuals with an extremely high risk of developing LOAD and individuals with an very low risk of developing LOAD.

Grünblatt et al. (“Aldehyde dehydrogenase (ALDH) in Alzheimer's and Parkinson's disease”, Journal Of Neural Transmission, vol. 123, no. 2, 9 Oct. 2014, pages 83-90) have discussed a link between genetic variants of the gene of ALDH2 and the risk for developing Alzheimer's disease.

Also, a link between variants of the gene of Nat2 and cognitive functions has been suggested (Selinski et al: “The ultra-slow NAT2*6A haplotypeis associated with reduced higher cognitive functions in an elderly study group”, Archives Of Toxicology, vol. 89, no. 12, 28 Nov. 2015, pages 2291-2303). However, a link to Alzheimer's disease could not be shown.

There have been attempts to improve ApoE4 based methods for prognosis of LOAD by combining determining ApoE4 with other biomarkers and using computer-based analysis of the identified features, as for example in WO2011/143574A2.

Furthermore, in US2009/260092A1 it has been suggested to combine the analysis of ApoE4 with determining SNPs of the gene GAB2 (GRB2-associated binding protein 2) in predicting the risk of late-onset Alzheimer's disease (LOAD). Similarly, US2015/073025A1 discloses SNPs of the TOMM40, which are used in combination with APOE4 in predicting the risk of LOAD. However, as is shown by the analysis of the comparative examples disclosed herein, no genetic interaction between ApoE4 and known SNPs of GAB2 could be identified. Furthermore, although the analysis disclosed herein is indicative of a slight genetic interaction of ApoE4 and TOMM40, the interaction of ApoE4 and the SNPs of the present invention allows a by far better prognosis of LOAD.

Furthermore, to date there are no effective therapeutic approaches for the treatment, prevention or deceleration of the development of LOAD, although it would be desirable that an individual that has been identified as developing LOAD should be treated.

In light of the prior art there remains a significant need in the art to provide additional and/or improved means for prognosis, risk assessment, risk stratification and/or diagnosis of a subject of developing LOAD. Furthermore, there is a need for medicaments for use in the treatment, prevention or deceleration of the development of LOAD in subjects that have been identified as being developing or being at risk of developing LOAD.

### SUMMARY OF THE INVENTION

In light of the prior art the technical problem underlying the present invention is the provision of alternative and/or improved means for prognosis, risk assessment, risk stratification and/or diagnosis of a subject of developing LOAD as well as the provision of a medicament for use in the treatment, prevention and/or deceleration of the development of LOAD in a subject at risk of developing LOAD.

This problem is solved by the features of the independent claims. Preferred embodiments of the present invention are provided by the dependent claims.

The invention therefore relates to a method for prognosis, risk assessment, risk stratification and/or diagnosis of a subject of developing late-onset Alzheimer's disease (LOAD), comprising
providing at least one sample isolated from said subject,
determining in said at least one sample the presence of
   ApoE4 and
   at least one of rs1799931(G), rs8192506(A), rs7653308(C), rs968529(C) and rs9658265(A),
wherein the presence or absence of
   ApoE4 and/or
   at least one of rs1799931(G), rs8192506(A), rs7653308(C), rs968529(C) and rs9658265(A) is indicative of a prognosis, a risk and/or a diagnosis of developing LOAD.

It has been known in the art that the presence of ApoE4, either as a heterozygous or homozygous allelic variant of the ApoE4 gene and/or as a protein isoform of the ApoE4 protein, in a subject is correlated with the development of LOAD and it has been suggested to provide a prognosis of developing LOAD on the basis of determining the presence of ApoE4. The present invention is based on the entirely surprising finding that the prognosis, risk assessment, risk stratification and/or diagnosis of a subject of developing LOAD can be improved by additionally determining in a sample from said patient, which may preferentially comprise genomic DNA, the presence of a specific base for at least one of five newly identified SNPs that are correlated with the development or absence of development of LOAD. Thereby it is unexpectedly possible by the method of the present invention to further stratify subjects in more specific risk groups as compared to methods of the state of the art that only determine the presence of ApoE4.

For example, when determining the presence of the ApoE4 allelic variant in a subject, said subject can either be ApoE4 negative, ApoE4 heterozygous, meaning that the subjects carries one ApoE4 allele, or the subject can be ApoE4 homozygous, meaning that the subjects carries two ApoE4 alleles. Depending on the configuration of the two ApoE4 alleles, a subject can be categorized into 3 risk groups, wherein it was believed that an ApoE4 negative person has a risk of about 30% to develop LOAD and the risk of ApoE4 heterozygous and ApoE4 homozygous subjects was considered to be about 55% and about 80%, respectively. However, these values might differ depending on the clinical study and the data set that was used to assess the respective risks.

By means of the method of the present invention, it is possible to further characterize the genetic predisposition of a subject to develop LOAD by determining the presence of at least one of rs1799931(G), rs8192506(A), rs7653308(C), rs968529(C) and rs9658265(A), allowing to further specify the risk of a subject of one of the three determined ApoE4 categories. Accordingly, the present invention provides a method for a more precise assessment of the risk of a subject for developing LOAD as compared to previous methods. The relatively heterogeneous groups of subjects as categorized by determining ApoE4 can be further subdivided according to the presence of one or more of rs1799931(G), rs8192506(A), rs7653308(C), rs968529(C) and rs9658265(A) to stratify the subjects into more homogenous categories that provide a better predictive certainty with respect to the risk of developing LOAD.

The method of the present invention has the advantage, that the determination of additional genetic features, namely the homozygous or heterozygous presence of one of rs1799931(G), rs8192506(A), rs7653308(C), rs968529(C) and rs9658265(A), represents an objective parameter that can be measured in an unbiased way. This represents a great advantage over previous methods that rely on parameters that are difficult to assess or are changing over time, such as dietary or smoking habits or the weight of a subject.

The present invention represents a great prospect with respect to patient management, since patients can be categorized with a better precision. Patients, relatives and medical personnel have improved certainty, which allows to start, provide or plan suitable preventive, therapeutic, care and/or social measures. Furthermore, it will allow to provide high risk patients with suitable preventive, prophylactic or therapeutic measures and/or medication, while low risk patients that might most likely not require such often very expensive treatments are not considered for these measures or medications.

Additionally, it is possible to more precisely stratify patients according to their risk categories on the basis of the method of the present invention, which can be a great advantage when testing new preventive, prophylactic or therapeutic measures, such as for example new medicaments, in the context of a clinical trial. Such trials often require a very high number of patients to be included in the trial due to the heterogeneity of the patients. Often, beneficial effects of newly developed medicaments cannot be identified due to the heterogeneity of the tested patients, although the medicament may have a positive effect on a subgroup of patients. Accordingly, the method of the present invention can be used to categorize and stratify patient groups for clinical trials, so that a more homogenous group patient that actually requires a certain treatment can be tested and the result is not blurred by inappropriate patients. Similarly, the method of the present invention can be used to decide whether a subject should be considered to receive a certain treatment of medication in a personalized medicine approach.

In embodiments, the invention relates to a method for prognosis, risk assessment, risk stratification and/or diagnosis of a subject of developing late-onset Alzheimer's disease (LOAD), comprising
providing at least one sample isolated from said subject,
determining in said at least one sample the presence of ApoE4 and at least one of
   rs1799931(G), and/or
   rs8192506(A), rs7653308(C), rs968529(C) and rs9658265(A),
wherein the presence or absence of ApoE4 and at least one of
   rs1799931(G), and/or
   rs8192506(A), rs7653308(C), rs968529(C) and rs9658265(A)
is indicative of a prognosis, a risk and/or a diagnosis of developing LOAD.

In embodiments, the invention relates to a method comprising determining in said at least one sample the presence of rs1799931(G).

Such embodiments are specifically preferred, because rs1799931(G) was surprisingly identified as risk factor for the development of LOAD. It was unexpectedly found that determining the presence or absence of this SNP, which was previously not described to play any role in the development of LOAD or any other neurodegenerative disease, allows a more precise stratification of subjects with respect to their risk of developing LOAD.

Preferably, in the context of the method of the present invention the presence of ApoE4 and/or rs1799931(G) correlates with an increased likelihood of developing LOAD. This embodiment of the present invention is particularly advantageous since it allows on the basis of the presence of rs1799931(G) to assign a higher risk or likelihood of developing LOAD to a subject. Accordingly, a carrier of rs1799931(G) can be categorized into a group of subjects with a higher risk of developing LOAD as compared to subjects that have the same allelic configuration with respect to the ApoE4 gene but do not carry rs1799931(G). Accordingly, the presence of rs1799931(G) in combination with the presence of ApoE4 indicates a higher risk of developing LOAD. Furthermore, for ApoE4 and rs1799931(G) the presence of two of the respective alleles (homozygous presence) may be associated with a higher risk than the presence of only one of the respective alleles (heterozygous presence). The presence of one or two alleles of rs1799931(G) in combination with either one or two alleles of ApoE4 or even in the absence of ApoE4 may be indicative of an increased risk of developing LOAD. Therein, the presence of two alleles of rs1799931(G) is correlated with a higher risk than the presence of only one allele of rs1799931(G). Accordingly, the group with the highest risk of developing LOAD according to this embodiment of the invention comprises homozygous carriers of both ApoE4 and rs1799931(G).

According to a further embodiment of the method of the invention, the presence of ApoE4 and/or rs1799931(G) is indicative of the development of LOAD. In the context of embodiments of the invention, the term “indicative of the development of LOAD” and “indicative of the absence of the development of LOAD” is intended as a measure of risk and/or likelihood. Preferably, the “indication” of the development or the absence of the development of LOAD is intended as a risk assessment, and is typically not to be construed in a limiting fashion as to point definitively to the absolute presence or absence of the development of LOAD. Accordingly, the term can be understood as indicating a low or high risk of the occurrence of LOAD, respectively.

Furthermore, in preferred embodiments of the invention the presence of two alleles of rs1799931(G) correlates with an increased likelihood of developing of LOAD. Furthermore, in preferred embodiment of the invention the presence of two alleles of rs1799931(G) is indicative of the development of LOAD. It is a particular advantage of this embodiment of the present invention that the presence of two alleles of rs1799931(G) in a subject allows to categorize a subject with a higher certainty as being at risk of developing LOAD as compared to categorizing the patient only on the basis of the presence of one allele of rs1799931(G).

When analyzing genomic data from LOAD patients, it was surprisingly found that the presence of rs1799931(G) on both alleles was strongly correlated with the development of LOAD, in particular in patients carrying one or two alleles of ApoE4. This strong correlation was completely unexpected and the method of the present invention implements this surprising finding to improve known methods of risk assessment and risk stratification to allow a more precise and accurate categorization of patients with respect to their risk of developing LOAD.

In embodiments, the invention relates to a method comprising determining in said at least one sample the presence of ApoE4 and rs1799931(G); or the presence of ApoE4 and all of rs8192506(A), rs7653308(C), rs968529(C) and rs9658265(A): or the presence of ApoE4, rs1799931(G) and all of rs8192506(A), rs7653308(C), rs968529(C) and rs9658265(A).

Importantly, the method of the invention can be carried out not only by determining by in addition to ApoE4 all of rs1799931(G) and rs8192506(A), rs7653308(C), rs968529(C) and rs9658265(A), but also by determining in addition to ApoE4 either only rs1799931(G) or only all of rs8192506(A), rs7653308(C), rs968529(C) and rs9658265(A). This is evident form the example of the invention shown below. rs1799931(G), similar to ApoE4, can be regarded as a risk factor for the development of LOAD, whereas the combination of rs8192506(A), rs7653308(C), rs968529(C) and rs9658265(A) can be regarded as a protective factor concerning the development of LOAD. Accordingly, by determining only one of these two factors in addition to ApoE4 it is already possible to provide a better risk assessment concerning the development of LOAD. However, by determining both, rs1799931(G) as an additional risk factor and rs8192506(A), rs7653308(C), rs968529(C) and rs9658265(A) as a protective factor, the method can be further improved.

In a further embodiment of the invention, the method comprises determining in said at least one sample the presence of rs8192506(A), rs7653308(C), rs968529(C) and rs9658265(A). It was entirely surprising, that determining the presence of these 4 SNPs, which have so far not been recognized as being linked with each other, and none of which has been reported to be correlated or inversely correlated with the development of LOAD, can provide information with respect to the development of LOAD in a subject, either independently or in combination with further markers or risk factors, such as ApoE4 and/or rs1799931(G).

In embodiments of the invention, the presence of rs8192506(A), rs7653308(C), rs968529(C) and rs9658265(A) correlates with a decreased likelihood of developing of LOAD. In embodiments of the invention, the presence of rs8192506(A), rs7653308(C), rs968529(C) and rs9658265(A) is indicative of the absence of the development of LOAD.

It was completely unexpected that the combined presence of rs8192506(A), rs7653308(C), rs968529(C) and rs9658265(A) is inversely correlated with the occurrence or development of LOAD in a subject. Accordingly, these SNPs can be regarded as a safety markers, meaning that the presence of these markers indicates a decreased risk of a subject to develop LOAD. This observation was entirely surprising and lacks any antecedent description in the art. Accordingly, the present invention that is implementing this finding is completely unexpected.

In a further preferred embodiment of the invention, the presence of two alleles of each of rs8192506(A), rs7653308(C), rs968529(C) and rs9658265(A) correlates with a decreased likelihood of developing of LOAD. In a further preferred embodiment of the invention, the presence of two alleles of each of rs8192506(A), rs7653308(C), rs968529(C) and rs9658265(A) is indicative of the absence of the development of LOAD.

By analyzing genetic data provided from a high number of LOAD patients and subjects that did not develop LOAD, it was found that the presence of two alleles of each of rs8192506(A), rs7653308(C), rs968529(C) and rs9658265(A) in the genome of a patient shows a strong inverse correlation with the development of LOAD. Surprisingly, this was in particular the case for subjects, that would otherwise be categorized as having a high risk of developing LOAD, because they are positive for risk factors that are positively correlated with the occurrence of LOAD such as ApoE4 or rs1799931(G). Accordingly, the combined occurrence of two alleles of each of rs8192506(A), rs7653308(C), rs968529(C) and rs9658265(A) can be regarded as a strongly protective marker, which indicates that a subject is less likely to develop LOAD.

In a further embodiment of the invention, the method comprises determining in said at least one sample the presence of rs7653308(C), rs968529(C) and rs9658265(A). In a further embodiment of the invention, the method comprises determining in said at least one sample the presence of rs8192506(A), rs968529(C) and rs9658265(A). In a further embodiment of the invention, the method comprises determining in said at least one sample the presence of rs8192506(A), rs7653308(C) and rs9658265(A). In a further embodiment of the invention, the method comprises determining in said at least one sample the presence of rs8192506(A), rs7653308(C) and rs968529(C).

In a further embodiment of the invention, the method comprises determining in said at least one sample the presence of rs8192506(A) and rs9658265(A). In a further embodiment of the invention, the method comprises determining in said at least one sample the presence of rs8192506(A) and rs968529(C). In a further embodiment of the invention, the method comprises determining in said at least one sample the presence of rs8192506(A) and rs7653308(C). In a further embodiment of the invention, the method comprises determining in said at least one sample the presence of rs7653308(C) and rs968529(C). In a further embodiment of the invention, the method comprises determining in said at least one sample the presence of rs7653308(C) and rs9658265(A). In a further embodiment of the invention, the method comprises determining in said at least one sample the presence of rs968529(C) and rs9658265(A).

In a further embodiment of the invention, the method comprises determining in said at least one sample the presence of rs1799931(G), rs8192506(A), rs7653308(C), rs968529(C) and rs9658265(A).

This embodiment of the present invention is particularly advantageous because therein all five SNPs that are either positively or negatively correlated with the development of LOAD, as disclosed herein for the first time, are determined together with the presence of ApoE4, which allows a sophisticated stratification of subjects with respect to their risk of developing LOAD. In the state of the art, there are only very limited objective markers which are correlated with LOAD, ApoE4 being the by far most relevant. However, on the basis of the configuration of the ApoE4 allelic variants, patients could only be stratified into 3 groups. By means of the present invention and in particular the preferred embodiment described above, patients can be categorized in a far more accurate way by taking into account the presence of rs1799931(G) and the combined presence of rs8192506(A), rs7653308(C), rs968529(C) and rs9658265(A), wherein each of these SNPs or markers can be either homozygous or heterozygous, which may result in a different risk group categorization.

According to a further embodiment, the method of the present invention comprises determining in said at least one sample the presence of at least one polymorphism in addition to ApoE4, wherein said additional polymorphism is in linkage disequilibrium with at least one of ApoE4, rs1799931(G), rs8192506(A), rs7653308(C), rs968529(C) and rs9658265(A).

According to a further embodiment, the method of the present invention comprises determining in said at least one sample the presence of at least one additional polymorphism, wherein said additional polymorphism is in linkage disequilibrium with at least one of ApoE4, rs1799931(G), rs8192506(A), rs7653308(C), rs968529(C) and rs9658265(A).

The term “linkage disequilibrium” relates to the non-random association of alleles, SNPs or polymorphisms, at different loci in a given population. Loci are said to be in linkage disequilibrium when the frequency of association of their different alleles is higher or lower than what would be expected if the loci were independent and associated randomly. Linkage disequilibrium may exist between alleles at different loci without any genetic linkage between them and independently of whether or not allele frequencies are in equilibrium (not changing with time).

For linkage disequilibrium (LD) structure examination D′ and r² measures can be used (Hill, W. G., and Robertson, A. (1968). Linkage disequilibrium in finite populations. Theoret. Appl. Genet. 38, 226-231). Analysis of measures can be performed using HAPLOVIEW Version 3.32 (https://www.broadinstitute.org/haploview/haploview; Barrett J C, Fry B, Mailer J, Daly M J. Haploview: analysis and visualization of LD and haplotype maps. Bioinformatics. 2005 Jan. 15), and blocks can defined using the confidence interval method described by Gabriel et al. (Gabriel, S. B. et al. (2002). The structure of haplotype blocks in the human genome. Science 296, 2225-2229. 2002).

In embodiments, the method of the invention comprises determining in said at least one sample the presence of ApoE4 and at least one of rs1799931(G), rs8192506(A), rs7653308(C), rs968529(C) and rs9658265(A), wherein at least one of rs1799931(G), rs8192506(A), rs7653308(C), rs968529(C) and rs9658265(A) is in linkage disequilibrium with at least one of the other markers of the present invention.

The embodiments of the present method comprising determining the presence of rs968529(C) or rs9658265(A) are particularly advantageous, because it was found that rs968529 and rs9658265 are in nearly perfect equilibrium. Accordingly, it is possible to conclude of the presence of one of rs968529(C) or rs9658265(A) about the presence of the other of rs968529(C) or rs9658265(A).

The presence of one or more additional linkage disequilibria between the polymorphisms of the method of the invention, or between one or more polymorphisms of the method of the invention with one or more additional polymorphisms may be identified. Identification of such an additional linkage disequilibrium may lead to embodiments of the present invention, wherein the determining of such an additional polymorphism can substitute the determining of a SNP of the method of the invention in the context of the presently claimed method. In embodiments of the invention, determining the presence of a polymorphism that is in linkage disequilibrium with one or more of the polymorphisms of the present invention, is equivalent to determining the presence of this one or more polymorphisms of the present invention. The polymorphisms of the present invention comprise ApoE4, rs1799931(G), rs8192506(A), rs7653308(C), rs968529(C) and rs9658265(A).

Accordingly, the present invention also relates to a method for prognosis, risk assessment, risk stratification and/or diagnosis of a subject of developing late-onset Alzheimer's disease (LOAD), comprising
providing at least one sample isolated from said subject,
determining in said at least one sample the presence of
   ApoE4 and/or one or more polymorphisms which are in linkage disequilibrium with ApoE4, and
   at least one of rs1799931(G), rs8192506(A), rs7653308(C), rs968529(C) and rs9658265(A), and/or one or more polymorphisms which are in linkage disequilibrium with one or more of rs1799931(G), rs8192506(A), rs7653308(C), rs968529(C) and rs9658265(A),
wherein the presence or absence of
   ApoE4 and/or the one or more polymorphisms which are in linkage disequilibrium with ApoE4, and/or
   at least one of rs1799931(G), rs8192506(A), rs7653308(C), rs968529(C) and rs9658265(A) and/or one or more polymorphisms which are in linkage disequilibrium with one or more of rs1799931(G), rs8192506(A), rs7653308(C), rs968529(C) and rs9658265(A), is indicative of a prognosis, a risk and/or a diagnosis of developing LOAD.

The present invention further relates to embodiments comprising
determining in said sample the presence of ApoE4, rs1799931(G), rs8192506(A), rs7653308(C), rs968529(C) and rs9658265(A), and
determining the likelihood of developing LOAD using the following formula: log odds=−0.8336156+A*0.3726496+0.5043521*B*A+−0.3261833*C, wherein:
   A=2 when two alleles of ApoE4 are present, A=1 when one allele of ApoE4 is present and A=0 when ApoE4 is not present,
   B=1 when two alleles of rs1799931(G) are present and otherwise B=0, and
   C=1 when two alleles of each of rs8192506(A), rs7653308(C), rs968529(C) and rs9658265(A) are present and otherwise C=0.

The present invention further relates to embodiments comprising
determining in said sample the presence of ApoE4, rs1799931(G), rs8192506(A), rs7653308(C), rs968529(C) and rs9658265(A), and
determining the likelihood of developing LOAD using the following formula: log odds=−0.833441+0.3890697*A+0.5277427*B+−0.3770894*C, wherein:
   A=2 when two alleles of ApoE4 are present, A=1 when one allele of ApoE4 is present and A=0 when ApoE4 is not present,
   B=1 when two alleles of rs1799931(G) are present and otherwise B=0, and
   C=1 when two alleles of each of rs8192506(A), rs7653308(C), rs968529(C) and rs9658265(A) are present and otherwise C=0.

By means of analyzing a high number of genetic information from LOAD patients and non-LOAD subjects it was possible to develop a formula that on the basis of the presence or absence of the markers described herein calculates a probability or likelihood of a subject to develop LOAD in the future. This formula allows to calculate the risk of a subject to develop LOAD with a higher accuracy than any method known in the art and, therefore, it represents a significant improvement over known technologies.

Moreover, embodiments of the present invention comprise additionally
informing the subject of the results of the method for prognosis, risk assessment, risk stratification and/or diagnosis of a subject of developing late-onset Alzheimer's disease (LOAD), and/or
classifying and/or stratifying the subject in the context of a clinical trial and/or with respect to a future treatment regime.

The method of the present invention may additionally comprise the stratification or classification of the subject of the method of the invention. The stratification or classification of the patients according to the method of the invention can be used to provide guidance with respect to the future treatment options and other consequences. Therefore, the method may additionally involve a step of informing the patient about the outcome of the test, since this result may have consequences for example on the future lifestyle of said subject. Furthermore, the method can involve the provision of guidance on modifying the lifestyle, medication and treatment on the basis of the result of the method of the invention, such as provision of specific dietary plans and/or medication options and regimes.

In embodiments of the invention, the method involves a treating the subject. The treatment can be initiated and selected on the basis of the result of the method of the invention. In case a subject has an increased risk of being diagnosed of developing LOAD, a specific treatment may be initiated. Such a treatment can involve preventive measures, symptomatic measures and/or curative measures. Treatments of LOAD to be carried out in the context of the method of the invention comprise, without limitation, administration of cholinesterase inhibitors, mematine, inhibitors of acetylcholinesterase, an antagonist of a receptor for the neurotransmitter glutamate, drugs from the psychiatric toolbox to control depression (antidepressants) and behavioral abnormalities; a modified diet, increased intellectual activities, cognitive stimulation, cognitive rehabilitation, reminiscence and life story work; measures to reduce the risk of cardiovascular symptoms, which are linked to LOAD, such as stopping smoking, reducing alcohol, a healthy and balanced diet including fruit and vegetables every day, exercising, blood pressure control.

Furthermore, the method of the invention comprises a method for prognosis, risk assessment, risk stratification and/or diagnosis of a subject of developing late-onset Alzheimer's disease (LOAD), comprising
having provided or obtained at least one sample isolated from said subject,
determining in said at least one sample the presence of ApoE4 and at least one of
   rs1799931(G), and/or
   rs8192506(A), rs7653308(C), rs968529(C) and rs9658265(A),
   wherein the presence or absence of ApoE4 and at least one of
   rs1799931(G), and/or
   rs8192506(A), rs7653308(C), rs968529(C) and rs9658265(A) is indicative of a prognosis, a risk and/or a diagnosis of developing LOAD,
carrying out a treatment of the subject.

In embodiments of the invention, the method additionally comprises determining one or more risk factors, such as sex, smoking habits, comorbidities and/or obesity. It is possible and potentially advantageous to further include such risk factors in the method of the present invention to further specify the risk of a subject of developing LOAD. It is considered that sociodemographic factors, lifestyle habits and other environmental factor play an important role in the development of LOAD. Accordingly, considering such factors in addition may provide a better prognosis for the development of LOAD.

The present invention further relates to a kit for carrying out the method of the invention, the kit comprising:
a computer program or a computer-readable media containing means for determining the likelihood of developing LOAD, and optionally
detection reagents for determining in a sample from a subject the presence of ApoE4 and at least one of rs1799931(G), rs8192506(A), rs7653308(C), rs968529(C) and rs9658265(A).

The kit of the present invention comprises a computer program which can be adapted for providing a prognosis, risk assessment, risk stratification and/or diagnosis of a subject of developing late-onset Alzheimer's disease (LOAD) on the basis of the presence of ApoE4 and the presence of at least one of rs1799931(G), rs8192506(A), rs7653308(C), rs968529(C) and rs9658265(A) in a sample from said subject.

The invention also relates to a kit for carrying out the method of the invention, the kit comprising:
a computer program or a computer-readable media adapted for providing a prognosis, risk assessment, risk stratification and/or diagnosis of a subject of developing late-onset Alzheimer's disease (LOAD) on the basis of the presence of ApoE4 and at least one of
   rs1799931(G), and/or
   rs8192506(A), rs7653308(C), rs968529(C) and rs9658265(A)
      in a sample from said subject, and optionally
detection reagents for determining in a sample from a subject the presence of ApoE4 and at least one of
   rs1799931(G), and/or
   rs8192506(A), rs7653308(C), rs968529(C) and rs9658265(A).

The invention further relates to a kit for carrying out the method of the invention, the kit comprising detection reagents for determining in a sample from a subject the presence of ApoE4 and at least one of rs1799931(G), and/or rs8192506(A), rs7653308(C), rs968529(C) and rs9658265(A). Such a kit may further comprise instructions on how to analyze and interpret the result of the detection reactions carried out using the detection reagents of the SNP for providing a prognosis, risk assessment, risk stratification and/or diagnosis of a subject of developing late-onset Alzheimer's disease (LOAD).

Such detection reagents can comprise SNP-specific PCR-primers, SNP-specific TaqMan probes and/or other suitable detection probes to be used in a nucleic acid amplification based detection method. Further reagents for detecting the presence of the SNPs of the invention can be designed or generated by the skilled person by using known methods of the art for detecting a specific SNP in a sample.

The different embodiments of the method of the present invention and features described therein as well as the associated advantages are herewith also disclosed in the context of the kit of the present invention. The advantages of the method of the invention and the advantages associated with features of embodiments of the method of the invention also apply to the kit of the present invention.

The present invention further relates to an inhibitor of the enzymatic activity of N-acetyltransferase 2 (Nat2) for use as a medicament in the treatment, prevention and/or deceleration of the development of LOAD in a subject at risk of developing LOAD or suffering from LOAD.

Furthermore, the present invention relates to an inhibitor of the enzymatic activity of N-acetyltransferase 2 (Nat2) for use as a medicament in the treatment, prevention and/or deceleration of the development of LOAD in a subject at risk of developing LOAD or suffering from LOAD, wherein the subject has been identified by employing the method of the present invention.

This aspect of the present invention is based on the surprising finding that patients at risk of developing LOAD as identified by the method of the present invention may be carriers of rs1799931(G). This particular SNP configuration is strongly correlated with an increased risk of LOAD as well as with a fast metabolizer phenotype of the enzyme Nat2, which is encoded by the gene harboring rs1799931. Surprisingly, inhibition of Nat2 and in particular the rapid metabolizer variant of Nat2 leads to deceleration and/or prevention of the development of LOAD in patients at risk of developing LOAD, wherein these patients have been identified by the method of the present invention.

Furthermore, the present invention relates to an inhibitor of the enzymatic activity of N-acetyltransferase 2 (Nat2) for use as a medicament in the treatment, prevention and/or deceleration of the development of LOAD in a subject at risk of developing LOAD, wherein the subject has been identified by employing the method of the present invention.

The method and kit of the present invention and the inhibitor of the enzymatic activity of N-acetyltransferase 2 (Nat2) for use as a medicament in the treatment, prevention and/or deceleration of the development of LOAD in a subject at risk of developing LOAD or suffering from LOAD are linked by the common new and inventive concept that rs1799931(G) is correlated with an increased risk of developing LOAD as well as with a rapid metabolizer phenotype of Nat2. The rapid metabolizer phenotype of Nat2 contributes to the pathogenesis of LOAD and contributes to the development of LOAD, wherein the development of LOAD and the risk of developing LOAD can be inhibited or decreased, respectively, by reverting the rapid metabolizer phenotype of Nat2 through specific inhibition.

Preferably, the inhibitor of the enzymatic activity of N-acetyltransferase 2 (NAT2) for use as a medicament in the treatment, prevention and/or deceleration of the development of LOAD in a subject at risk of developing LOAD or suffering from LOAD is an inhibitor of a rapid metabolizer variant of Nat2.

According to a preferred embodiment of the invention, the inhibitor of the enzymatic activity of NAT2 is N-acetyl-para-aminophenol.

The present invention further encompasses a method for the production of a pharmaceutical composition for use as a medicament in the treatment or prevention of LOAD in a subject at risk of developing LOAD or suffering from LOAD, comprising
a method for identifying an inhibitor of the enzymatic activity of NAT2,
mixing the identified compound or a derivative or homologue thereof with a pharmaceutically acceptable carrier,
wherein the subject has been identified by employing the method of the present invention.

The present invention further encompasses a method for the production of a pharmaceutical composition for use as a medicament in the treatment or prevention of LOAD in a subject at risk of developing LOAD, comprising
a method for identifying an inhibitor of the enzymatic activity of a rapid metabolizer variant of NAT2,
mixing the identified compound or a derivative or homologue thereof with a pharmaceutically acceptable carrier,
wherein the subject has been identified by employing the method of the present invention.

### DETAILED DESCRIPTION OF THE INVENTION

All cited documents of the patent and non-patent literature are hereby incorporated by reference in their entirety.

The present invention is directed to a method for prognosis, risk assessment, risk stratification and/or diagnosis of a subject of developing late-onset Alzheimer's disease (LOAD).

As used herein, the terms “patient” and “subject” are used interchangeably and may relate to a vertebrate. In the context of the present invention, the terms include both humans and animals, particularly mammals, and other organisms.

The present invention relates to methods, kits and compounds for use as a medicament in the context of late-onset Alzheimer's disease (LOAD). LOAD in the sense of the present invention refers to the most common form of AD, which mostly occurs late in life, preferentially in subjects with an age of 60 years or more. It is thought that multiple genetic and environmental risk factors may be involved in LOAD pathogenesis, and overall impairment in Aβ clearance is probably a major contributor to disease development. The development of LOAD may therefore depend on the presence or absence of certain risk factors or environmental influences and accordingly is difficult to predict.

“Prognosis” relates to the prediction of the later occurrence of LOAD in a subject or the development of LOAD in said subject. Prognosis may further refer to calculating a specific risk of the subject to develop LOAD based on the method of the present invention.

In the present invention, the terms “risk assessment” and “risk stratification” relate to the grouping of subjects into different risk groups according to their further prognosis of developing LOAD. Risk assessment also relates to stratification for applying preventive and/or therapeutic measures. Examples of the risk stratification may be the classification of a patient into specific risk groups according to their risk levels, such as for example low, intermediate and high risk levels. Such risk levels or risk groups may be determined by performing the method of the present invention, optionally in combination with the assessment of additional risk factors, which may be considered for the risk stratification of a subject.

As used herein, “diagnosis” relates to the identification, recognition and (early) detection of a clinical condition of a subject, preferably LOAD. Also the assessment of the severity of the clinical condition and the state of progression or full development of the conditions may be encompassed by the term “diagnosis”.

The term “developing” in the context of developing a clinical condition and specifically developing LOAD refers to a state, wherein a subject may already suffer from LOAD, although the symptoms are still very mild or non-detected of non-detectable, and also a state, wherein the disease has been diagnosed and is still developing in the sense of progressing or continuing. A person at risk of developing LOAD may be a person who does not yet suffer from LOAD or at least does not display any symptoms of the disease, but may develop LOAD in the future, wherein the risk of developing LOAD may be increased for the subject, for example due to the presence or absence of one or more markers of the present invention.

As used herein, the term “sample” is a biological sample that is obtained or isolated from the patient or subject. “Sample” as used herein may, e.g., refer to a sample of bodily fluid or tissue obtained for the purpose of diagnosis, prognosis, or evaluation of a subject of interest, such as a patient. Preferably herein, the sample is a sample of a bodily fluid, such as blood, serum, plasma, cerebrospinal fluid, urine, saliva, sputum, pleural effusions, cells, a cellular extract, a tissue sample, a tissue biopsy, a stool sample and the like. The sample may be any kind of sample isolated from a subject that comprises genomic material form said subject or is suitable for the isolation of genomic material, such as genomic DNA.

As used herein, terms such as “marker” or “prognostic marker” or “biological marker” are used interchangeably and relate to measurable and quantifiable biological markers (e.g., specific protein or protein isoform or a fragment thereof, nucleic acid molecules comprising a specific sequence or sequence variant or SNP) which serve as indices for health- and physiology-related assessments, such as a disease/disorder/clinical condition risk, preferably a risk of developing or having LOAD. A marker or biomarker is defined as a characteristic that can be objectively measured and evaluated as an indicator of normal biological processes, pathogenic processes, or pharmacologic responses to a therapeutic intervention. Biomarkers may be measured in a sample.

“Genetic variants” in the context of this application refers to genetic differences both within and among populations. There may be multiple variants of any given gene in the human population (alleles), leading to polymorphisms. The terms “polymorphism” and “single nucleotide polymorphism” (SNP) are used herein interchangeably and relate to a nucleotide sequence variation occurring when a single nucleotide in the genome or another shared sequence differs between members of species or between paired chromosomes in an individual. A SNP can also be designated as a mutation with low allele frequency greater than about 1% in a defined population. Single nucleotide polymorphisms according to the present application may fall within coding sequences of genes, non-coding regions of genes or the intronic regions between genes.

A “SNP” or single-nucleotide polymorphism is a variation in a single nucleotide that occurs at a specific position in the genome, where each variation is present to some appreciable degree within a population (e.g. >1%). For example, at a specific base position in the human genome, the C nucleotide may appear in most individuals, but in a minority of individuals, the position is occupied by an A. This means that there is a SNP at this specific position, and the two possible nucleotide variations—C or A—are said to be alleles for this position. SNPs underlie differences in our susceptibility to disease; a wide range of human diseases, e.g. sickle-cell anemia, β-thalassemia and cystic fibrosis result from SNPs. The severity of illness and the way the body responds to treatments are also manifestations of genetic variations.

Single-nucleotide polymorphisms may fall within coding sequences of genes, non-coding regions of genes, or in the intergenic regions. SNPs within a coding sequence do not necessarily change the amino acid sequence of the protein that is produced, due to degeneracy of the genetic code. SNPs in the coding region are of two types: Synonymous SNPs that do not affect the protein sequence, and nonsynonymous SNPs that change the amino acid sequence of protein.

The nonsynonymous SNPs are of two types: missense and nonsense. SNPs that are not in protein-coding regions may still affect gene splicing, transcription factor binding, messenger RNA degradation, or the sequence of noncoding RNA. Gene expression affected by this type of SNP is referred to as an eSNP (expression SNP) and may be upstream or downstream from the gene.

A specific SNP is defined and identifiable through the rs-number, which is the reference number of a SNP, such as the rs-numbers of the present invention (e.g. rs1799931, rs8192506, rs7653308, rs968529, rs9658265, rs429358, rs7412). The “rs” prefix is added to all of the genetic variations officially recognized in the US National Institute of Health's database, dbSNP, which is part of the National Center for Biotechnology Information (NCBI) collection of websites and is also used by other databases, such as SNPedia (www.snpedia.com). Further details are known to the skilled person and can be found in McEntyre J, Ostell J, editors. Bethesda (Md.): National Center for Biotechnology Information (US) (in particular Chapter 5, The Single Nucleotide Polymorphism Database (dbSNP) of Nucleotide Sequence Variation).

In general, in the context of the present invention, when naming a SNP by using the rs-reference-number and indicating in brackets a single letter, this single letter refers to the base or nucleotide to be found at the location of the respective SNP. For example, when referring to determining the presence of rs429358(T), this means that it is determined whether at the location of the SNP rs429358 a thymine (T) is present. As known to the person skilled in the art, adenine is indicated as A, cytosine as C, guanine as G, thymine as T and uracil as U.

Apolipoprotein E (ApoE) is a protein that is encoded by the ApoE gene. The ApoE protein becomes a lipoprotein when combined with fat lipoprotein ApoE is a very low-density lipoprotein, responsible in part for removing cholesterol from the bloodstream. Variations in ApoE affect cholesterol metabolism, which in turn alter your chances of having heart disease and in particular a heart attack or a stroke. Variations in ApoE are also associated with altered odds of having Alzheimer's disease and other diseases. There are three relatively common allelic variants of ApoE, as defined by two SNPs, rs429358 and rs7412 known as ApoE-ε2, ApoE-ε3, and ApoE-ε4. The proteins produced by these genes are called ApoE2, ApoE3, and ApoE4.

As used herein, the term “ApoE4” refers to both the protein variant (or isoform) and the allelic variant of the gene on the nucleic acid level. Accordingly, when referring to “determining in said at least one sample the presence of ApoE4”, this relates to determining the presence of the ApoE4 allelic variant of the gene on a nucleic acid molecule in a sample or the presence of isoform 4 of the protein ApoE in a sample or both.

The most common variant overall is the “standard” ApoE-ε3, and therefore more people inherited one ApoE-ε3 from each parent than any other of the possible pairs of variants. Note that each of these types can have additional changes too, so there are different subtypes as well.

TABLE 1

| rs429358 | rs7412 | Name |
|---|---|---|
| C | T | ε1 |
| T | T | ε2 |
| T | C | ε3 |
| C | C | ε4 |

The single letters in the table refer to the base or nucleotide to be found at the location of the respective SNP in the respective allelic variant.

TABLE 2

| | Allele 1 | Allele 2 |
|---|---|---|
| Apo-ε/ε | rs429358/rs7412 | rs429358/rs7412 |
| Apo-ε1/ε1 | C/C | T/T |
| Apo-ε1/ε2 | C/T | T/T |
| Apo-ε1/ε3 | C/T | C/T |
| Apo-ε1/ε4 | C/C | C/T |
| Apo-ε2/ε2 | T/T | T/T |
| Apo-ε2/ε3 | T/T | C/T |
| Apo-ε2/ε4 | C/T | C/T |
| Apo-ε3/ε3 | T/T | C/C |
| Apo-ε3/ε4 | C/T | C/C |
| Apo-ε4/ε4 | C/C | C/C |

The corresponding protein isoform are ApoE2 (Cys112, Cys158), ApoE3 (Cys112, Arg158), and ApoE4 (Arg112, Arg158).

Although ApoE status is technically defined by these two SNPs, rs429358 and rs7412, a SNP in the adjacent ApoC1 gene, rs4420638, is co-inherited with ApoE and thus predictive of it, as known to the skilled person (Nat Genet. 2007 January; 39(1):17-23. Bertram L1, McQueen M B, Mullin K, Blacker D, Tanzi R E.). Accordingly, in the context of the present invention the presence of ApoE4 may be determined or estimated by determining rs4420638. The present invention may also comprise determining rs4420638 in said sample.

Determining the presence of rs8192506(A) refers to determining whether at the location of the SNP rs8192506 there is a A present. rs8192506 is located in the DBI gene, which is associated with a missense in the protein sequence of diazepam binding inhibitor or known as acyl-coA-binding-protein. Diazepam binding inhibitor is regulated by hormones and is involved in lipid metabolism and the displacement of beta-carbolines and benzodiazepines, which modulate signal transduction at the type A gamma-aminobutyric acid (GABA) receptors located in brain synapses (Neurology 1990 40:632-5).

Determining the presence of rs7653308(C) refers to determining whether at the location of the SNP rs7653308 there is a C present. rs7653308 is located in an intronic region of the FGF12 gene encoding Fibroblast growth factor 12, which is involved in the development of the nervous system (Proc Natl Acad Sci USA 1996 93: 9850-7).

Determining the presence of rs968529(C) refers to determining whether at the location of the SNP rs968529there is a C present. rs968529 is located in an intronic region of the ALDH2 gene encoding Aldehyde dehydrogenase, which is involved in neurodegeneration (J Neural Transm (Vienna) 2016 123:83-90).

Determining the presence of rs9658265(A) refers to determining whether at the location of the SNP rs9658265 there is a A present. rs9658265 is located in an intronic region of the NOS1 gene encoding Nitric oxide synthase (neuronal), which has been shown to be involved in nitro oxidative stress (J Alzheimer Dis 2007 11:207-18).

Determining the presence of rs1799931(G) refers to determining whether at the location of the SNP rs17799931 there is a G present. rs1799931 is a SNP in the NAT2 gene which is associated with a missense in the protein sequence of the protein N-Acetyltransferase 2.

According to the present invention, the term “indicate” in the context of “indicative of the development of LOAD” and “indicative of the absence of the development of LOAD” is intended as a measure of risk and/or likelihood. Preferably, the “indication” of the development or the absence of the development of LOAD is intended as a risk assessment and is typically not to be construed in a limiting fashion as to point definitively to the absolute presence or absence of the development of LOAD. Therefore, the term “indicative of the absence of the development of LOAD” or “indicative of the development of LOAD” can be understood as indicating a low or high risk of the occurrence of LOAD, respectively.

Here, odds are an expression of relative probabilities. Generally quoted as the odds in favor. The odds (in favor) of an event or a proposition is the ration of the probability that the event will happen to the probability that the event will not happen. In other words: Odds describes how many times an event occurs, for each time, it does not occur.

Log odds are the same as odds, only in logarithmic space. The coefficient of a logistic regression, a regression model where the dependent variable is categorical, are basically represented in log odds. Log odds can be used to calculate the probability using the formula:

Probability=exp(x)/1+exp(x)

Determining the presence or absence of a marker, such as a specific isoform of a ApoE protein, or a SNP or polymorphism or genetic variant, preferably one or more of rs1799931(G), rs8192506(A), rs7653308(C), rs968529(C) and rs9658265(A) can be performed by any analysis method suitable for and capable of determining the presence of proteins, protein variants or isoforms, and/or nucleotides and nucleotide sequences in nucleic acid molecules. The particular method used is not important for performing the method of the present invention. Once a biological sample from a subject has been obtained (e.g., a bodily fluid, such as urine, saliva, plasma, serum, or a tissue sample, such as a buccal tissue sample or a buccal cell) detection of a protein variant or nucleic acid sequence variation or allelic variant SNP is typically undertaken. Virtually any method known to the skilled artisan can be employed.

Those skilled in the art will readily recognize that the analysis of the nucleotides present according to the method of the invention in an individual nucleic acid molecule can be done by any method or technique capable of determining nucleotides present in a polymorphic site. As it is obvious in the art, the nucleotides present in the polymorphic markers can be determined from either nucleic acid strand or from both strands. Perhaps the most direct method is to actually determine the sequence of either genomic DNA or cDNA and compare these sequences to the known alleles SNPs of the gene. This technology is quite common and is well known. Any of a variety of methods that exist for detecting sequence variations may be used in the methods of the invention.

Other possible commercially available methods exist for the high throughput SNP identification not using direct sequencing technologies, for example, Illumina's Veracode Technology, Taqman® SNP Genotyping Chemistry and KASPar SNP genotyping Chemistry. A variation the direct sequence determination method is the Gene Chip™ method available from Affymetrix. Alternatively, robust and less expensive ways of detecting DNA sequence variation are also commercially available. For example, Perkin Elmer adapted its TAQman Assay™ to detect sequence variation. Orchid BioSciences has a method called SNP-IT™ (SNP-identification Technology) that uses primer extension with labeled nucleotide analogs to determine which nucleotide occurs at the position immediately 3′ of an oligonucleotide probe, the extended base is then identified using direct fluorescence, an indirect colorimetric assay, mass spectrometry, or fluorescence polarization. Sequenom uses a hybridization capture technology plus MALDI-TOF (Matrix Assisted Laser Desorption/Ionization—Time-of-Flight mass spectrometry) to detect SNP genotypes with their MassARRAY™ system. Promega provides the READIT™ SNP/Genotyping System (U.S. Pat. No. 6,159,693). In this method, DNA or RNA probes are hybridized to target nucleic acid sequences. Probes that are complementary to the target sequence at each base are depolymerized with a proprietary mixture of enzymes, while probes which differ from the target at the interrogation position remain intact. The method uses pyrophosphorylation chemistry in combination with luciferase detection to provide a highly sensitive and adaptable SNP scoring system. Third Wave Technologies has the Invader OS™ method that uses proprietary Cleavaseg enzymes, which recognize and cut only the specific structure formed during the Invader process. Invader OS relies an linear amplification of the signal generated by the invader process, rather than an exponential amplification of the target. The Invader OS assay does not utilize PCR in any part of the assay. In addition, there are a number of forensic DNA testing labs and many research labs that use gene-specific PCR, followed by restriction endonuclease digestion and gel electrophoresis (or other size separation technology) to detect restriction fragment length polymorphisms (RFLPs).

In various embodiments of any of the above aspects, the presence or absence of the SNPs is identified by amplifying or failing to amplify an amplification product from the sample. Polynucleotide amplifications are typically template-dependent. Such amplifications generally rely on the existence of a template strand to make additional copies of the template. Primers are short nucleic acids that are capable of priming the synthesis of a nascent nucleic acid in a template-dependent process, which hybridize to the template strand. Typically, primers are from ten to thirty base pairs in length, but longer sequences can be employed. Primers may be provided in double-stranded and/or single-stranded form, although the single-stranded form generally is preferred. Often, pairs of primers are designed to selectively hybridize to distinct regions of a template nucleic acid, and are contacted with the template DNA under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids containing one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

The determination of protein sequences or the presence of protein isoforms in a sample from a subject may be performed by, for example, mass spectrometry or antibody-mediated techniques such as ELISA and western blot. “Mass spectrometry” or “MS” refers to an analytical technique to identify compounds by their mass. In order to enhance the mass resolving and mass determining capabilities of mass spectrometry, the samples can be processed prior to MS analysis.

Accordingly, the invention relates to MS detection methods that can be combined with immuno-enrichment technologies, methods related to sample preparation and/or chromatographic methods, preferably with liquid chromatography (LC), more preferably with high performance liquid chromatography (HPLC) or ultra high performance liquid chromatography (UHPLC). Sample preparation methods comprise techniques for lysis, fractionation, digestion of the sample into peptides, depletion, enrichment, dialysis, desalting, alkylation and/or peptide reduction. However, these steps are optional. The selective detection of analyte ions may be conducted with tandem mass spectrometry (MS/MS). Tandem mass spectrometry is characterized by mass selection step (as used herein, the term “mass selection” denotes isolation of ions having a specified m/z or narrow range of m/z″s), followed by fragmentation of the selected ions and mass analysis of the resultant product (fragment) ions. The skilled person is aware how quantify the level of a marker in the sample by mass spectrometric methods. For example, relative quantification “rSRM” or absolute quantification can be employed as described above.

The term “risk factor” as used herein refers to parameters and factors, such for example a biomarker, such as a genetic or a protein marker that may be present in a sample isolated form a subject, or sociodemographic and/or clinical characteristics, which may affect the risk of developing LOAD, including for example a family history of LOAD, dementia, neurodegeneration and/or neurological diseases, body mass index, sex, age, smoking habits, comorbidities and/or obesity.

The invention further relates to kits, the use of the kits and methods wherein such kits are used. The invention relates to kits for carrying out the herein above and below provided methods. The herein provided definitions, e.g. provided in relation to the method, also apply to the kits of the invention. In particular, the invention relates to a kit for prognosis, risk assessment, risk stratification and/or diagnosis of a subject of developing late-onset Alzheimer's disease (LOAD), or a kit for carrying out the method of the invention, comprising:
a computer program or a computer-readable media containing means for determining the likelihood of developing LOAD, and optionally
detection reagents for determining in a sample from a subject the presence of ApoE4 and at least one of rs1799931(G), rs8192506(A), rs7653308(C), rs968529(C) and rs9658265(A).

As explained above, the determination of the various markers can be performed using a great variety of methods and the term “detection reagents” for determining the presence of a marker refers to any kind of reagents that may be provided together with the computer program or computer-readable containing means for determining the likelihood of developing LOAD.

The kit may additionally comprise items useful for obtaining a sample, such as a blood sample, for example the kit may comprise a container, wherein said container comprises a device for attachment of said container to a canula or syringe, is a syringe suitable for blood isolation, exhibits an internal pressure less than atmospheric pressure, such as is suitable for drawing a pre-determined volume of sample into said container, and/or comprises additionally detergents, chaotropic salts, ribonuclease inhibitors, chelating agents, such as guanidinium isothiocyanate, guanidinium hydrochloride, sodium dodecylsulfate, polyoxyethylene sorbitan monolaurate, RNAse inhibitor proteins, and mixtures thereof, and/or A filter system containing nitro-cellulose, silica matrix, ferromagnetic spheres, a cup retrieve spill over, trehalose, fructose, lactose, mannose, poly-ethylen-glycol, glycerol, EDTA, TRIS, limonene, xylene, benzoyl, phenol, mineral oil, anilin, pyrol, citrate, and mixtures thereof.

The methods of the present invention may in part be computer-implemented. For example, the step of determining or calculating the likelihood of developing LOAD on the basis of the determining of the markers of the present invention may be performed by a computer system or a computer program. In the context of the kit of the present invention, such a computer program or means for calculating the likelihood of developing LOAD may be provided on a computer-readable medium. In the computer-system or computer program, the determined presence of the respective markers can be combined with other markers and/or parameters of the subject in order to calculate a score, which is indicative for the diagnosis, prognosis, risk assessment and/or risk stratification.

For example, the determined presence or absence of the respective markers may be entered (either manually by a health professional or automatically from the device(s) in which the respective markers have been determined) into the computer-system or program. The computer-system can be directly at the point-of-care (e.g. primary care, ICU or ED) or it can be at a remote location connected via a computer network (e.g. via the internet, or specialized medical cloud-systems, optionally combinable with other IT-systems or platforms such as hospital information systems (HIS)). Typically, the computer-system will store the parameters (e.g. presence/absence of a marker or parameters such as age, weight, sex, etc.) on a computer-readable medium and calculate a score or likelihood or risk level of developing LOAD. The result may be displayed and/or printed for the user (typically a health professional such as a physician). Alternatively or in addition, the associated prognosis, diagnosis, assessment, treatment guidance, patient management guidance or stratification will be displayed and/or printed for the user (typically a health professional such as a physician).

In one embodiment of the invention, a software system can be employed, in which a machine learning algorithm is evident, preferably to identify patients at risk of developing LOAD. A machine learning approach can be trained on a random forest classifier using EHR data (such as labs, presence/absence of markers, biomarker expression, vitals, and demographics) from patients. Machine learning is a type of artificial intelligence that provides computers with the ability to learn complex patterns in data without being explicitly programmed, unlike simpler rule-based systems.

N-acetyltransferase 2, also known as NAT2, is an enzyme which in humans is encoded by the NAT2 gene. The NAT2 isozyme functions to both activate and deactivate arylamine and hydrazine drugs and carcinogens. Drugs reported to be metabolized by NAT2 include isoniazid, sulfadimidine, hydralazine, dapsone, procaine amide, sulfapyridine, nitrazepam and some sulfa drugs. Polymorphisms in NAT2 are also associated with higher incidences of cancer and drug toxicity.

Polymorphisms in NAT2 are responsible for the N-acetylation polymorphism in which human populations segregate into rapid, intermediate, and slow acetylator phenotypes. It takes two slow metabolizer alleles to give rise to a slow metabolizer phenotype, or to put it another way, the rapid metabolizer allele is dominant to the slow metabolizer, and you therefore only need one to be a rapid metabolizer.

Generally, with respect to NAT2, individuals are therefore classified as rapid metabolizers if they have one or more NAT2*4 alleles, and slow metabolizers only if they carry two slow metabolizer variants. The alleles themselves are effectively haplotypes composed of several NAT2 SNPs, most typically assigned according to the status of the following seven SNPs:
rs1801279, aka G191A
rs1041983, aka C282T
rs1801280, aka T341C
rs1799929, aka C481T
rs1799930, aka G590A
rs1208, aka A803G
rs1799931, aka G857A

NAT2*4 is considered to be the wild-type allele, and the exemplar rapid metabolizer; consists of the first nucleotide shown in the “aka” (also known as) names listed above for these seven NAT2 SNPs, i.e. an allele in question is NAT2*4 if it is rs1801279(G) and rs1041983(C) and rs1801280(T) and so on.

Almost all of the remaining common alleles are slow metabolizers, such as:
NAT2*5A: 341C+481T, i.e. rs1801280(C)+rs1799929(T)
NAT2*5B: 341C+481T+803G, i.e. rs1801280(C)+rs1799929(T)+rs1208(G)
NAT2*5C: 341C+803G, i.e. rs1801280(C)+rs1208(G)
NAT2*5D: 341C, i.e. rs1801280(C)
NAT2*5E: 341C+590A, i.e. rs1801280(C)+rs1799930(A)
NAT2*5G: 282T+341C+481T+803G, i.e. rs1041983(T)+rs1801280(C)+rs1799929(T)+rs1208(G)
NAT2*5J: 282T+341C+590A, i.e. rs1041983(T)+rs1801280(C)+rs1799930(A)
NAT2*6A: 282T+590A, i.e. rs1041983(T)+rs1799930(A)
NAT2*6B: 590A (only), i.e. no variation compared to NAT2*4 except rs1799930(A)
NAT2*6C: 282T+590A+803G, i.e. rs1041983(T)+rs1799930(A)+rs1208(G)
NAT2*6E: 481T+590A, i.e. rs1799929(T)+rs1799930(A)
NAT2*7A: 857A (only), i.e. rs1799931(A)
NAT2*7B: 282T+857A, i.e. rs1041983(T)+rs1799931(A)
NAT2*14A: 191A, i.e. rs1801279(A)
NAT2*14B: 191A+282T, i.e. rs1801279(A)+rs1041983(T)
NAT2*14C: 191A+341C+481T+803G, i.e. rs1801279(A)+rs1801280(C)+rs1799929(T)+rs1208(G)
NAT2*14D: 191A+282T+590A, i.e. rs1801279(A)+rs1041983(T)+rs1799930(A)
NAT2*14E: 191A+803G, i.e. rs1801279(A)+rs1208(G)
NAT2*14F: 191A+341C+803G, i.e. rs1801279(A)+rs1801280(C)+rs1208(G)
NAT2*14G: 191A+282T+803G, i.e. rs1801279(A)+rs1041983(T)+rs1208(G)

However, there are also a few rapid (i.e. normal) metabolizer variants as well, such as:
NAT211A: 481T, i.e. rs1799929(T)
NAT2*12A: 803G, i.e. rs1208(G)
NAT2*12B: 282T+803G, i.e. rs1041983(T)+rs1208(G)
NAT2*12C: 481T+803G, i.e. rs1799929(T)+rs1208(G)
NAT2*13: 282T (only), i.e. rs1041983(T)

The present invention further relates to inhibitors or N-acetyltransferase 2 (Nat2) for use as a medicament in the treatment, prevention and/or deceleration of the development of LOAD in a subject at risk of developing LOAD.

Different inhibitor of the enzymatic activity of N-acetyltransferase 2 (Arylamine N-acetyltransferase 2, Nat2) are well known in the art as well as different methods or for identifying an inhibitor of the enzymatic activity of NAT2.

Examples of inhibitors of Nat2 comprise, without limitation, CHEMBL458614 (C10H512NO2S2; Bioorg. Med. Chem., (2009) 17:2:905), CHEMBL3262074 (C21H13NO4; Bioorg. Med. Chem., (2014) 22:11:3030), CHEMBL3262073(C24H15NO4; Bioorg. Med. Chem., (2014) 22:11:3030), and N-acetyl-para-aminophenol (also known as acetaminophen or APAP, paracetamol, Int J Immunopathol Pharmacol 2016 29:17-22; Pharmacogenetics 1998 8:553-559) and derivatives thereof. The methods used for identifying the respective inhibitors or their inhibitory function for Nat2 are described in the referenced publication, and are therefore known and easily accessible for the person skilled in the art. Multiple further inhibitors of Nat2 are listed for example in the ChEMBL database (https://www.ebi.ac.uk/chembl), demonstrating that Nat2 inhibitors represent a known and well described group of inhibitory molecules.

N-acetyl-para-aminophenol, also known as paracetamol, acetaminophen or APAP, is a medication used to treat pain and fever. However, it has also been described as an inhibitor of Nat2 and in particular of the rapid metabolizer variant Nat2*4.

The present invention encompasses treatment of a patient or subject by administering a therapeutically effective amount of an inhibitor of Nat2. Such administering can be performed, for example, once, a plurality of times, and/or over one or more extended periods. Repeated injections over time (e.g., daily, weekly, monthly, quarterly, half-yearly or yearly) may be necessary. Such administering is also preferably performed using an admixture a pharmaceutically acceptable carrier. Pharmaceutically acceptable carriers or pharmaceutically acceptable salts, which are well known to those skilled in the art.

The term “pharmaceutically acceptable salt” refers to salts or esters of the compounds described herein prepared by conventional means that include basic salts of inorganic and organic acids, including but not limited to hydrochloric acid, hydrobromic acid, sulfuric acid, phosphoric acid, methanesulfonic acid, ethanesulfonic acid, malic acid, acetic acid, oxalic acid, tartaric acid, citric acid, lactic acid, fumaric acid, succinic acid, maleic acid, salicylic acid, benzoic acid, phenylacetic acid, mandelic acid and the like. Any chemical compound recited in this specification may alternatively be administered as a pharmaceutically acceptable salt thereof. Also included are acidic salts of inorganic and organic bases, including but not limited to sodium, potassium, ammonium, triethylamine and the like.

“Pharmaceutically acceptable salts” are also inclusive of the free acid, base, and zwitterionic forms. Descriptions of suitable pharmaceutically acceptable salts can be found in Handbook of Pharmaceutical Salts, Properties, Selection and Use, Wiley VCH (2002). For therapeutic use, salts of the compounds are those wherein the counter-ion is pharmaceutically acceptable. However, salts of acids and bases which are non-pharmaceutically acceptable may also find use, for example, in the preparation or purification of a pharmaceutically acceptable compound.

Another aspect of the disclosure includes pharmaceutical compositions prepared for administration to a subject and which include a therapeutically effective amount of one or more of the compounds disclosed herein. The therapeutically effective amount of a disclosed compound will depend on the route of administration, the species of subject and the physical characteristics of the subject being treated. Specific factors that can be taken into account include disease severity and stage, weight, diet and concurrent medications. The relationship of these factors to determining a therapeutically effective amount of the disclosed compounds is understood by those of skill in the art.

Pharmaceutical compositions for administration to a subject can include at least one further pharmaceutically acceptable additive such as carriers, thickeners, diluents, buffers, preservatives, surface active agents and the like in addition to the molecule of choice. Pharmaceutical compositions can also include one or more additional active ingredients such as antimicrobial agents, anti-inflammatory agents, anesthetics, and the like. The pharmaceutically acceptable carriers useful for these formulations are conventional. Remington's Pharmaceutical Sciences, by E. W. Martin, Mack Publishing Co., Easton, Pa., 19th Edition (1995), describes compositions and formulations suitable for pharmaceutical delivery of the compounds herein disclosed.

In general, the nature of the carrier will depend on the particular mode of administration being employed. For instance, parenteral formulations usually contain injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

In accordance with the various treatment methods of the disclosure, the compound can be delivered to a subject in a manner consistent with conventional methodologies associated with management of the disorder for which treatment or prevention is sought. In accordance with the disclosure herein, a prophylactically or therapeutically effective amount of the compound and/or other biologically active agent is administered to a subject in need of such treatment for a time and under conditions sufficient to prevent, inhibit, and/or ameliorate a selected disease or condition or one or more symptom(s) thereof.

“Administration of” and “administering a” compound should be understood to mean providing a compound, a prodrug of a compound, or a pharmaceutical composition as described herein. The compound or composition can be administered by another person to the subject (e.g., intravenously) or it can be self-administered by the subject (e.g., tablets).

Any references herein to a compound for use as a medicament in the treatment of a medical condition also relate to a method of treating said medical condition comprising the administration of a compound, or composition comprising said compound, to a subject in need thereof, or to the use of a compound, composition comprising said compound, in the treatment of said medical condition.

Dosage can be varied by the attending clinician to maintain a desired concentration at a target site (for example, the lungs or systemic circulation). Higher or lower concentrations can be selected based on the mode of delivery, for example, trans-epidermal, rectal, oral, pulmonary, or intranasal delivery versus intravenous or subcutaneous delivery. Dosage can also be adjusted based on the release rate of the administered formulation, for example, of an intrapulmonary spray versus powder, sustained release oral versus injected particulate or transdermal delivery formulations, and so forth.

The present invention also relates to a method of treatment of subjects suffering from the various medical conditions disclosed herein. The method of treatment comprises preferably the administration of a therapeutically effective amount of a compound disclosed herein to a subject in need thereof.

In the context of the present invention, the term “medicament” refers to a drug, a pharmaceutical drug or a medicinal product used to diagnose, cure, treat, or prevent disease. It refers to any substance or combination of substances presented as having properties for treating or preventing disease. The term comprises any substance or combination of substances, which may be used in or administered either with a view to restoring, correcting or modifying physiological functions by exerting a pharmacological, immunological or metabolic action, or to making a medical diagnosis. The term medicament comprises biological drugs, small molecule drugs or other physical material that affects physiological processes.

According to the present invention, the term “treatment” refers to a therapeutic intervention that ameliorates a sign or symptom of a disease or pathological condition after it has begun to develop. As used herein, the term “ameliorating”, with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, an improvement in the overall health or well-being of the subject, or by other parameters well known in the art that are specific to the particular disease.

The present invention encompasses both treatment and prophylactic treatment of a subject. A “prophylactic” treatment is a treatment administered to a subject who does not exhibit signs of a disease or exhibits only early signs for the purpose of decreasing the risk of developing pathology.

### EXAMPLES

The invention is further described by the following examples. These are not intended to limit the scope of the invention, but represent preferred embodiments of aspects of the invention provided for greater illustration of the invention described herein.

### Example of the Invention

Introduction: Late-onset Alzheimer's Disease (LOAD) has an important genetic component. In addition to the reported ApoE4 gene, new genetic variants in associated with this pathology are highly needed for better LOAD risk prediction. The aim of this study was to determine whether a set of polygenic genetic variants selected by us improves the ability of ApoE4 to predict the risk of Late-onset Alzheimer's Disease.

Methods: We investigated a genome-wide association study (GWAS) of 1.895 LOAD cases and 1.971 controls. A set of 50 SNPs reported to be involved in lipid metabolism was selected. Then, based on the 50 SNPs from the lipid metabolism and the ApoE SNPs, models were built by the R package Glmnet Vignette (https://web.stanford.eduhhastie/glmnet/glmnet_alpha.html) to for the better prediction of LOAD (Jerome Friedman, Trevor Hastie and Rob Tibshirani. (2008). Regularization Paths for Generalized Linear Models via Coordinate Descent. Journal of Statistical Software, Vol. 33(1), 1-22 Feb. 2010.; Noah Simon, Jerome Friedman, Trevor Hastie and Rob Tibshirani. (2011). Regularization Paths for Cox's Proportional Hazards Model via Coordinate Descent. Journal of Statistical Software, Vol. 39(5) 1-13.; Robert Tibshirani, Jacob Bien, Jerome Friedman, Trevor Hastie, Noah Simon, Jonathan Taylor, Ryan J. Tibshirani. (2010). Strong Rules for Discarding Predictors in Lasso-type Problems. Journal of the Royal Statistical Society: Series B (Statistical Methodology), 74(2), 245-266.; Noah Simon, Jerome Friedman and Trevor Hastie (2013). A Blockwise Descent Algorithm for Group-penalized Multiresponse and Multinomial Regression. (in arXiv, submitted)). The procedure was repeated until a model could be selected that predicts more accurately the LOAD risk.

The SNPs rs968529 and rs 9658265 are in close distance on chromosome 12. Therefore, potential linkage disequilibrium (LD) was investigated: r-square of <0.001 and D′ of 0.018 were calculated indicating that rs968529 and rs9658265 are nearly in perfect equilibrium.

Results: Based on the mathematical model relevant SNPs were identified and a formula for the risk evaluation was obtained.

The positions of the identified SNPs were checked (Table 3):

TABLE 3

| SNP | Allele | Gene | Function | Position | Product |
|---|---|---|---|---|---|
| rs1799931 | A/G | NAT2*4 | missense | 8:18400860 | N-Acteyltransferase 2 |
| rs8192506 | A/C/G | DBI | missense | 2:119372265 | Diazepam binding protein |
| rs7653308 | A/C/T | FGF12 | intron | 3:192445262 | Fibroblast growth factor 12 |
| rs968529 | C/T | ALDH2 | intron | 12:111796564 | Aldehyde dehydrogenase |
| rs9658265 | A/G | NOS1 | intron | 12:117340065 | Nitric oxide synthase |

The SNPs rs968529 and rs9658265 are in close distance on chromosome 12. Therefore, potential linkage disequilibrium (LD) was investigated: r-square of <0.001 and D′ of 0.018 were calculated indicating that rs968529 and rs9658265 are nearly in perfect equilibrium.

The formula obtained on the basis of the mathematical model and the identified SNPs is as follows:

log odds=−0.8333441+0.3890697*A+0.5277427*B+−0.3770894*C, wherein:
A=2 when two alleles of ApoE4 are present, A=1 when one allele of ApoE4 is present and A=0 when ApoE4 is not present,
B=1 when two alleles of rs1799931(G) are present and otherwise B=0, and
C=1 when two alleles of each of rs8192506(A), rs7653308(C), rs968529(C) and rs9658265(A) are present and otherwise C=0.

Based on the above formula, the log odds for the probability of developing LOAD were calculated for every possible SNP combination (Table 4).

TABLE 4
Log odds for the probability of developing LOAD.

| | ApoE4 | ApoE4 | ApoE4 |
|---|---|---|---|
| | negative | heterozygous | homozygous |
| No other SNP | −0.8333441 | −0.4442744 | −0.0552047 |
| rs1799931 GG | −0.3056014 | +0.0834683 | +0.5829474 |
| rs1799931 GG + | −0.6826908 | −0.2936211 | +0.205858 |
| rs8192506 AA | | | |
| rs7653308 CC | | | |
| rs968529 CC | | | |
| rs9658265 AA | | | |
| rs8192506 AA | −1.2104335 | −0.8213638 | −0.4322941 |
| rs7653308 CC | | | |
| rs968529 CC | | | |
| rs9658265 AA | | | |

Next, we refrain to calculate the odds from the determined log odds. Instead, the late-onset Alzheimer's Disease (LOAD) frequency was calculated in analyzed genome-wide association data of 1.895 LOAD cases and 1.971 healthy controls for every combination based on following formula to get a better proof of our analysis:

LOAD frequency=number of carriers in cases/number of carriers in cases and controls

The result is summarized in the following Table 5:

TABLE 5
Calculated LOAD frequency.

| | ApoE4 | ApoE4 | ApoE4 |
|---|---|---|---|
| | negative | heterozygous | homozygous |
| No other SNP | 30% | 50% | 60% |
| rs1799931 GG | 50% | 60% | 90% |
| rs1799931 GG + | 25% | 50% | 70% |
| rs8192506 AA | | | |
| rs7653308 CC | | | |
| rs968529 CC | | | |
| rs9658265 AA | | | |
| rs8192506 AA | 20% | 25% | 50% |
| rs7653308 CC | | | |
| rs968529 CC | | | |
| rs9658265 AA | | | |

The risk groups defined by the log odds formula correlate exactly with the LOAD frequency calculated from the case control study.

Additionally, the LOAD frequency was analogously calculated only for the ApoE4 marker displaying the more precise prediction of the SNP combination identified herein compared to the single marker of ApoE4 (Table 6):

TABLE 6
Calculated LOAD frequency using only ApoE4.

| | ApoE4 | ApoE4 | ApoE4 |
|---|---|---|---|
| | negative | heterozygous | homozygous |
| LOAD | 30% | 55% | 80% |
| frequency (%) | | | |

Based on these findings the value of our prediction was analyzed regarding true negative, false negative, false positive, and true positive (Table 7):

TABLE 7

| True state | Prediction | |
|---|---|---|
| | No LOAD | LOAD |
| No LOAD | True Negative (TN) | False Positive (FP) |
| LOAD | False Negative (FN) | True Positive (TP) |

Here, the values for accuracy, precision, recall (sensitivity), and true negative rate were calculated as follows (Table 8):

TABLE 8
Accuracy, precision, recall, and true negative
rate for the method of the invention.

| | |
|---|---|
| Accuracy = (TP + TN)/(TP + TN + FP + FN): | 0.78 |
| Precision = (TP/(FP + TP): | 0.85 |
| Recall (Sensitivity) = TP/(FN + TP): | 0.57 |
| True negative rate = TN/(TN + FP): | 0.93 |

Compared with the single ApoE4 marker, we calculated the following LOAD frequencies and values for accuracy, precision, recall, and true negative rate (Table 9):

TABLE 9
Accuracy, precision, recall, and true
negative rate when using only ApoE4.

| | ApoE4 | ApoE4 | ApoE4 |
|---|---|---|---|
| | negative | heterozygous | homozygous |
| LOAD | 30% | 55% | 80% |
| frequency (%) | | | |

| | |
|---|---|
| Accuracy = (TP + TN)/(TP + TN + FP + FN): | 0.63 |
| Precision = (TP/(FP + TP): | 0.75 |
| Recall (Sensitivity) = TP/(FN + TP): | 0.12 |
| True negative rate = TN/(TN + FP): | 0.97 |

Discussion: We demonstrate that the selected interacting genetic profile significantly improves the prediction of the risk of Late-onset Alzheimer's Disease (LOAD). The accuracy and precision of our prediction is significantly better than the accuracy and precision for the single ApoE4 marker. Additionally, the recall (sensitivity) is dramatically improved with our polygenic biomarker (0.57) compared to this one from ApoE4 with 0.12. The genetic profile in clinical practice will improve the diagnosis, prevention, and hopefully leads to the optimal treatment of Late-onset Alzheimer's Disease (LOAD)

### Comparative Examples

Analysis of an interaction between ApoE4 and genetic variants of GAB2

A corresponding analysis as described above in the example of the invention was performed for ApoE4 and genetic variants of GAB2. In the analyzed data there are the following SNPs, which can be used as tags for the GAB2 gene:
rs642378
rs10501429
rs10793339
rs7936158
rs4388909
rs2277277

Models for APOE4 and one or more of the above SNPs were created to make predictions about the occurrence of LOAD, but it was not possible to achieve better results than with ApoE4 alone.

In a stepwise regression approach, in which features that do not improve the model drop out successively (“stepwise”) the above-mentioned GAB2 SNPs drop out of the model. Only APOE4 remains.

In conclusion, no interaction between GAB2 and ApoE4 could be identified that would allow a better prediction than only APOE4.

Analysis of an Interaction Between ApoE4 and Genetic Variants of TOMM40

A corresponding analysis as described above in the example of the invention was performed for ApoE4 and an identified genetic variant of TOMM40. In the analyzed data the SNP rs892117 was identified as a tag for the SNP rs10524523 of TOMM40 mentioned in the state of the art.

A model for ApoE4 and rs892117 was created to make predictions about the occurrence of LOAD. The predictions are a slightly better than the prediction made on the basis of APOE4 alone. In a stepwise regression approach, rs892117 remained in the model with the following performance:
Accuracy: 0.6814159
Precision: 0.6585366
Recall: 0.5510204
True negative rate: 0.6888889

In conclusion, the performance of the models of APOE4 and rs892117 indicates an interaction that can predict disease better than APOE4 alone. However, the performance is weaker than with the SNPs of the invention as evident from Table

1. A method for prognosis, risk assessment, risk stratification and/or diagnosis of a subject of developing late-onset Alzheimer's disease (LOAD), comprising:
providing at least one sample isolated from said subject,
determining in said at least one sample the presence of ApoE4 and at least one of:
rs1799931(G), and/or
rs8192506(A), rs7653308(C), rs968529(C) and rs9658265(A),
wherein the presence or absence of ApoE4 and at least one of:
rs1799931(G), and/or
rs8192506(A), rs7653308(C), rs968529(C) and rs9658265(A)
is indicative of a prognosis, a risk and/or a diagnosis of developing LOAD.

2. The method according to claim 1, comprising determining in said at least one sample the presence of rs1799931(G).

3. The method according to claim 1, comprising determining in said at least one sample the presence of rs8192506(A), rs7653308(C), rs968529(C) and rs9658265(A).

4. The method according to claim 1, wherein the presence of ApoE4 and/or rs1799931(G) correlates with an increased likelihood of developing LOAD.

5. The method according to claim 1, wherein the presence of ApoE4 and/or rs1799931(G) is indicative of the development of LOAD.

6. The method according to claim 1, wherein the presence of rs8192506(A), rs7653308(C), rs968529(C) and rs9658265(A) is indicative of the absence of the development of LOAD.

7. The method according to claim 1, wherein the presence of two alleles of rs1799931(G) is indicative of the development of LOAD.

8. The method according to claim 1, wherein the presence of two alleles of each of rs8192506(A), rs7653308(C), rs968529(C) and rs9658265(A) is indicative of the absence of the development of LOAD.

9. The method according to claim 1, comprising determining in said at least one sample the presence of at least one polymorphism in addition to ApoE4, wherein said additional polymorphism is in linkage disequilibrium with at least one of ApoE4, rs1799931(G), rs8192506(A), rs7653308(C), rs968529(C) and rs9658265(A).

10. The method according to claim 1, comprising
determining in said sample the presence of ApoE4, rs1799931(G), rs8192506(A), rs7653308(C), rs968529(C) and rs9658265(A), and
determining the likelihood of developing LOAD using the following formula:
log odds=−0.8333441+0.3890697*A+0.5277427*B+−0.3770894*C, wherein:
A=2 when two alleles of ApoE4 are present, A=1 when one allele of ApoE4 is present and A=0 when ApoE4 is not present,
B=1 when two alleles of rs1799931(G) are present and otherwise B=0, and
C=1 when two alleles of each of rs8192506(A), rs7653308(C), rs968529(C) and rs9658265(A) are present and otherwise C=0.

11. The method according to claim 1, comprising additionally
informing the subject of the results of the method for prognosis, risk assessment, risk stratification and/or diagnosis of a subject of developing late-onset Alzheimer's disease (LOAD), and/or
classifying and/or stratifying the subject in the context of a clinical trial and/or with respect to a future treatment regime.

12. The method according to claim 1, comprising additionally determining one or more risk factors, such as sex, smoking habits, comorbidities and/or obesity.

13. A kit for carrying out the method of claim 1, comprising:
a computer program or a computer-readable media adapted for providing a prognosis, risk assessment, risk stratification and/or diagnosis of a subject of developing late-onset Alzheimer's disease (LOAD) on the basis of the presence of ApoE4 and at least one of:
rs1799931(G), and/or
rs8192506(A), rs7653308(C), rs968529(C) and rs9658265(A) in a sample from said subject, and optionally
detection reagents for determining in a sample from a subject the presence of ApoE4 and at least one of:
rs1799931(G), and/or
rs8192506(A), rs7653308(C), rs968529(C) and rs9658265(A).

14. A method of treatment and/or deceleration of the development of LOAD in a subject at risk of developing LOAD, the method comprising:
identifying in the subject a prognosis, risk assessment, risk stratification and/or a diagnosis of developing late-onset Alzheimer's disease (LOAD) by employing the method of claim 1; and,
administering to the subject an inhibitor of the enzymatic activity of a rapid metabolizer variant of N-acetyltransferase 2 (Nat2), wherein the inhibitor is N-acetyl-para-aminophenol.

15. A method for the production of a pharmaceutical composition for use as a medicament in the treatment or prevention of LOAD in a subject at risk of developing LOAD, the method comprising:
identifying an inhibitor of the enzymatic activity of NAT2, and
mixing the identified compound or a derivative or homologue thereof with a pharmaceutically acceptable carrier.
